(19) United States
(12) Patent Application Publication
(10) Pub. No.: US 2008/0069778 A1
(43) Pub. Date: Mar. 20, 2008

(54) POLYMER-ENCAPSULATED MICROSPHERES

(75) Inventor: Nikhil Pargaonkar, Tucson, AZ (US)

Correspondence Address: DALE F. REGELMAN, CHANDLER & UDALL, LLP, 4801 E. BROADWAY BLVD #400, TUCSON, AZ, 85711-3609, US

(73) Assignee: IMARX THERAPEUTICS, INC., Tucson, AZ (US)

(21) Appl. No.: 11/858,763

(22) Filed: Sep. 20, 2007

Related U.S. Application Data
(60) Provisional application No. 60/826,367, filed on Sep. 20, 2006.

(51) Int. Cl. A61K 49/22; A61K 9/127

(52) U.S. Cl. 424/9.51; 424/450; 424/9.52

(57) ABSTRACT

A polymer-encapsulated phospholipid microsphere, comprising a plurality of lipid molecules disposed in a liquid and arranged to define an enclosed space, and one or more polymers partially or completely encapsulating that enclosed space. The one or more polymers may be functionalized with either a therapeutic agent and/or a targeting ligand either before, or after, deposition of the polymer onto the phospholipid microsphere.

### CROSS REFERENCE TO RELATED APPLICATIONS

This Application claims priority from a U.S. Provisional Application having Ser. No. 60/826,367, filed Sep. 20, 2006.

### FIELD OF THE INVENTION

It is known in the art to utlize ultrasound contrast agents in combination with traditional medical sonography. Ultrasound contrast agents comprise gas-filled microbubbles that are administered intravenously to the systemic circulation. Such microbubbles have a high degree of echogenicity, which is the ability of an object to reflect the ultrasound waves. The echogenicity difference between the gas in the microbubbles and the soft tissue surroundings of the body is immense.

Thus, ultrasonic imaging using microbubble contrast agents enhances the ultrasound backscatter, or reflection of the ultrasound waves, to produce a unique sonogram with increased contrast due to the high echogenicity difference. Contrast-enhanced ultrasound can be used to image blood perfusion in organs, measure blood flow rate in the heart and other organs, and has other applications as well.

### SUMMARY OF THE INVENTION

Applicant's invention comprises a polymer-encapsulated phospholipid microsphere. The microsphere comprises a plurality of lipid molecules arranged to defining an enclosed space, and one or more polymers partially or completely encapsulating that enclosed space. The one or more polymeric layers may be functionalized with either a therapeutic agent and/or a targeting ligand either before, or after, deposition onto the phospholipid microsphere.

### BRIEF DESCRIPTION OF THE DRAWINGS

The invention will be better understood from a reading of the following detailed description taken in conjunction with the drawings in which like reference designators are used to designate like elements, and in which:

FIG. 1 shows the surface charges of Applicant's unmodified microspheres and microspheres comprising a first polymer layer using Malvern zeta potentials;

FIG. 2 shows the surface charge of Applicant's unmodified microspheres, microspheres comprising a first polymer, and microspheres comprising a first polymer and a second polymer, using Malvern zeta potentials;

FIG. 3 shows a fluorescence analysis of Applicant's unmodified microspheres, microspheres comprising a first polymer, and microspheres comprising a first polymer and a second polymer;

FIG. 4 shows a particle size distribution for Applicant's unmodified microspheres;

FIG. 5 shows a particle size distribution for Applicant's microspheres comprising a first polymer;

FIG. 6A is a cross-sectional block diagram view of Applicant's unmodified microsphere comprising a contiguous surface;

FIG. 6B is a cross-sectional view of Applicant's unmodified microsphere showing a plurality of lipid molecules disposed in a liquid and arranged to define an enclosed space;

FIG. 7A shows a polymer comprising PLL completely encapsulating a spherical phospholipid;

FIG. 7B shows a polymer comprising PLL partially encapsulating a spherical phospholipid;

FIG. 7C shows the encapsulated microsphere of FIG. 7A functionalized with fluorescein moieties;

FIG. 7D shows the partially encapsulated microsphere of FIG. 7B functionalized with fluorescein moieties;

FIG. 8A is a cross-sectional block diagram view of Applicant's microsphere encapsulated with a first polymer and further encapsulated with a second polymer, wherein that second polymer comprises a plurality of pendent carboxylate moieties;

FIG. 8B shows the encapsulated microsphere of FIG. 8A, wherein the second polymer is amido-derivatized to comprise a plurality of pendent therapeutic agent moieties;

FIG. 8C shows the encapsulated microsphere of FIG. 8A, wherein the second polymer is ester-derivatized to comprise a plurality of pendent therapeutic agent moieties;

FIG. 8D shows the encapsulated microsphere of FIG. 8A, wherein the second polymer is amido-derivatized to comprise a plurality of pendent targeting ligand moieties;

FIG. 8E shows the encapsulated microsphere of FIG. 8A, wherein the second polymer is ester-derivatized to comprise a plurality of pendent targeting ligand moieties;

FIG. 8F shows the encapsulated microsphere of FIG. 8A, wherein the second polymer is ester-derivatized to comprise a plurality of pendent targeting ligand moieties in combination with a plurality of pendent therapeutic agent moieties;

FIG. 8G shows the encapsulated microsphere of FIG. 8A, wherein the second polymer is amido-derivatized to comprise a plurality of pendent targeting ligand moieties;

FIG. 9A is a cross-sectional block diagram view of Applicant's microsphere partially encapsulated with a first polymer and further partially encapsulated with a second polymer, wherein that second polymer comprises a plurality of pendent carboxylate moieties;

FIG. 9B shows the encapsulated microsphere of FIG. 9A, wherein the second polymer is amido-derivatized to comprise a plurality of pendent therapeutic agent moieties;

FIG. 9C shows the encapsulated microsphere of FIG. 9A, wherein the second polymer is ester-derivatized to comprise a plurality of pendent therapeutic agent moieties;

FIG. 9D shows the encapsulated microsphere of FIG. 9A, wherein the second polymer is amido-derivatized to comprise a plurality of pendent targeting ligand moieties;

FIG. 9E shows the encapsulated microsphere of FIG. 9A, wherein the second polymer is ester-derivatized to comprise a plurality of pendent targeting ligand moieties;

FIG. 9F shows the encapsulated microsphere of FIG. 9A, wherein the second polymer is ester-derivatized to comprise a plurality of pendent targeting ligand moieties in combination with a plurality of pendent therapeutic agent moieties;

FIG. 9G shows the encapsulated microsphere of FIG. 9A, wherein the second polymer is amido-derivatized to comprise a plurality of pendent targeting ligand moieties;

FIG. 10A is a cross-sectional block diagram view of Applicant's microsphere encapsulated with a first polymer, and further encapsulated with a second polymer, and further encapsulated with a colloidal gel;

FIG. 10B shows the encapsulated microsphere of FIG. 10A, wherein a plurality of therapeutic agents are releaseably disposed in the colloidal gel;

FIG. 10C is a cross-sectional block diagram view of Applicant's microsphere encapsulated with a first polymer and further encapsulated with a colloidal gel;

FIG. 10D shows the encapsulated microsphere of FIG. 10C, wherein a plurality of therapeutic agents are releaseably disposed in the colloidal gel;

FIG. 11A is a cross-sectional block diagram view of Applicant's microsphere partially encapsulated with a first polymer, and further partially encapsulated with a second polymer, and further encapsulated with a colloidal gel;

FIG. 11B shows the encapsulated microsphere of FIG. 10A, wherein a plurality of therapeutic agents are releaseably disposed in the colloidal gel;

FIG. 11C is a cross-sectional block diagram view of Applicant's microsphere partially encapsulated with a first polymer and further encapsulated with a colloidal gel;

FIG. 11D shows the encapsulated microsphere of FIG. 10C, wherein a plurality of therapeutic agents are releaseably disposed in the colloidal gel;

### DETAILED DESCRIPTION OF THE PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Applicant's invention comprises a polymer-encapsulated microsphere. By “polymer encapsulated microsphere,” Applicant means a plurality of lipid-like molecules arranged to define an enclosed space, wherein that enclosed space is partially or completely encapsulated by one or more polymers.

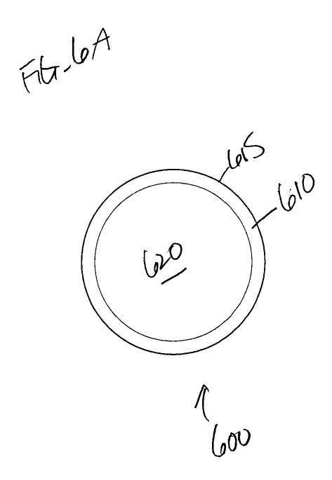

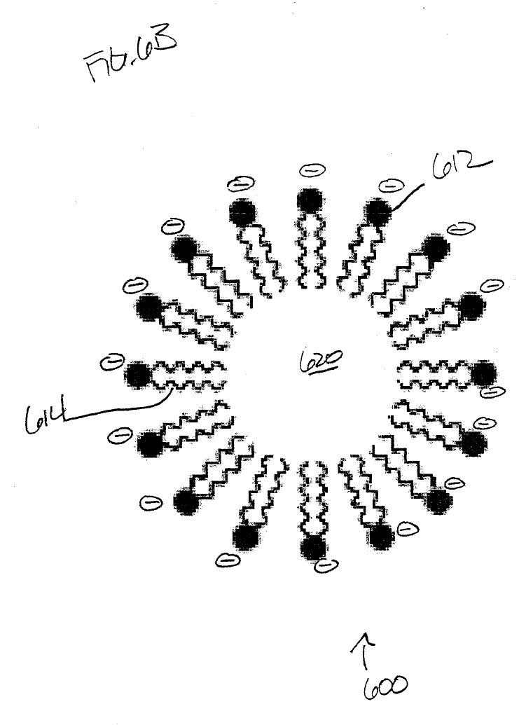

Referring now to FIGS. 6A and 6B, Applicant's acoustically active lipospheres composition comprises a plurality of microspheres 600 disposed in a liquid carrier system. By “microsphere,” Applicant means a material comprising at least one internal void 620. In certain embodiments, Applicants' microspheres comprise a plurality of phosphorus-containing compounds. Those phosphorus-containing compounds form lipid-like structures 610 in an aqueous medium. References herein to “lipids” refer to any combination of Applicants' plurality of phosphorus-containing compounds.

In any given microsphere, the lipids may be in the form of a monolayer or bilayer, and the mono- or bilayer lipids may be used to form one or more mono- or bilayers. In the case of more than one mono- or bilayer, the mono- or bilayers are generally concentric. The microspheres described herein include such entities commonly referred to as liposomes, micelles, bubbles, microbubbles, vesicles, and the like. Thus, the lipids may be used to form a unilamellar microsphere (comprised of one monolayer or bilayer), an oligolamellar microsphere (comprised of about two or about three monolayers or bilayers) or a multilamellar microsphere (comprised of more than about three monolayers or bilayers). In certain embodiments, the internal void 620 of the microsphere 600 is, partially or completely filled with a gas selected from the groups consisting of a fluorine-containing gas, a perfluorocarbon gas such as and without limitation perfluoropropane or perfluorobutane, a hydrofluorocarbon gas, sulfur hexafluoride, and mixture thereof.

In certain embodiments, Applicant's plurality of phosphorus-containing compounds comprises dipalmitoylphosphatidylethanolaminepolyethylene glycol (“DPPE-PEG”), dipalmitoylphosphatidylcholine (“DPPC”), and dipalmitoylphosphatidic acid (“DPPA”). As those skilled in the art will appreciate, each of Applicant's phosphorus-containing compounds is structurally similar to naturally-occurring lipid/phosolipid materials.

As those skilled in the art will further appreciate, lipids comprise a polar, hydrophilic, head 612 and one to three nonpolar, hydrophobic, tails 614. Phospholipids comprise materials having a hydrophilic head comprising a negatively charged phosphate group. For purposes of clarity and illustration, the illustration below does not show a positively charged counterion associated with the negatively charged phosphate group. 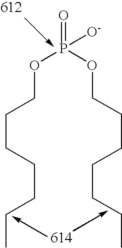
As a result, surface 615 (FIG. 6A) of microsphere 610 comprises a negative charge.

### Preparation of Microspheres 600

In certain embodiments, Applicant's method further provides a plurality of carbon-containing liquids and a plurality of salts. In certain embodiments, Applicant's plurality of carbon-containing liquids includes propylene glycol and glycerol. In certain embodiments, Applicant's plurality of salts includes sodium chloride, sodium phosphate monobasic, sodium phosphate dibasic.

In certain embodiments, Applicant's method forms a first mixture comprising the plurality of phosphorus-containing compounds in a first solvent, wherein that first solvent comprises one or more carbon atoms, and wherein that first solvent is water soluble, and wherein that first solvent does not comprise water.

In certain embodiments, Applicant's first mixture comprises a solution. In certain embodiments, Applicant's first solvent is infinitely water soluble. In certain embodiments, Applicant's first solvent comprises a polyol. In certain embodiments, Applicant's first solvent comprises propylene glycol. In certain embodiments, Applicant's first solvent consists essentially of propylene glycol.

Applicant's method forms a second mixture comprising a plurality of inorganic salts in a second solvent. In certain embodiments, Applicant's second mixture comprises a solution. In certain embodiments, Applicant's second solvent is water soluble. In certain embodiments, Applicant's second solvent is infinitely water soluble. In certain embodiments, Applicant's second solvent comprises water in combination with a carbon-containing liquid. In certain embodiments, that carbon-containing liquid comprises glycerol.

Applicant's method then combines the mixture comprising the plurality of phosphorus-containing compounds with the inorganic salt mixture to form Applicant's microsphere-forming composition. In certain embodiments, Applicant's microsphere-forming composition has a pH between about 5 and about 8. In certain embodiments, Applicant's microsphere-forming composition has a pH of about 6.5.

The following example is presented to further illustrate to persons skilled in the art how to make and use the invention. This example is not intended as a limitation, however, upon the scope of the invention.

### EXAMPLE 1

1. Dispose 100 mL of propylene glycol in a first vessel;

2. Place first vessel in an oil bath maintained at 60° C.±5° C.;

3. Add 60 milligrams of DPPA to the heated propylene glycol;

4. After dissolution of the DPPA, add 540 milligrams of DPPC to the heated propylene glycol solution;

5. After dissolution of the DPPA, add 400 milligrams of DPPE-PEG5000 to the heated propylene glycol solution;

6. After dissolution of the DPPE-PEG5000, stir heated propylene glycol solution using a Silverson high-speed stirrer at 3500 RMP for 5 minutes;

7. Dispose 850 mL of water in a second vessel;

8. Place second vessel in an water bath maintained at 60° C.±5° C.;

9. Add 50 mL of glycerol to heated water in second vessel;

10. Mix water/glycerol mixture using a magnetic stir bar for about 15 minutes;

11. Add 4.87 grams of sodium chloride to heated water/glycerol mixture;

12. Add 2.34 grams of sodium phosphate monobasic to the heated sodium chloride/water/glycerol mixture;

13. Add 2.16 grams of sodium phosphate dibasic to the heated sodium phosphate monobasic/sodium chloride/water/glycerol mixture;

14. Stir aqueous mixture until dissolution of all added salts;

15. Add the contents of the first vessel to the heated second vessel with stirring to form a lipid suspension.

### Polymeric Surface Charge Modification of Microspheres

Applicant has found that the ability to modify the surface charge of the microspheres enables the non-invasive incorporation of one or more therapeutic agents and/or one or more targeting ligands over the surface of those microspheres. Referring once again to FIGS. 6A and 6B, microsphere 600 comprises a negative charge on outer surface 615. In certain embodiments Applicant disposes a first polymeric layer over all or a portion of outer surface 615 of microsphere 600. In certain embodiments, that first polymeric layer comprises a plurality of pendant groups, wherein some or all of those pendant groups comprise a positive charge.

In certain embodiments that first polymeric layer comprises Poly/Lysine (“PLL”) I. 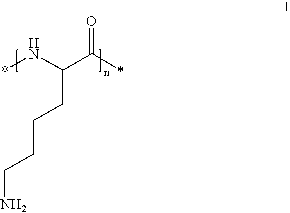
In certain embodiments, Applicant disposes a second polymeric layer over all or a portion of the first polymeric layer. In certain embodiments, that second polymeric layer comprises Poly Glutamic Acid (“PGA”) II.

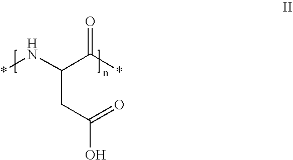

In still other embodiments, Applicant disposes a third gelatinous layer over the second polymeric layer. In certain embodiments, one or more therapeutic agents are releaseably disposed in that gelatinous layer.

Initial tests were conducted to determine the solubility of PLL and PGA in Applicants' lipid solutions. Both PLL and PGA are individually soluble in lipid solution. Applicant has found, however, that when mixed in equal proportions PLL and PGA form a precipitate in lipid solution. In addition, Applicant has found that gelatin, a specific hydrogel described hereinbelow, is soluble in Applicant's lipid solutions.

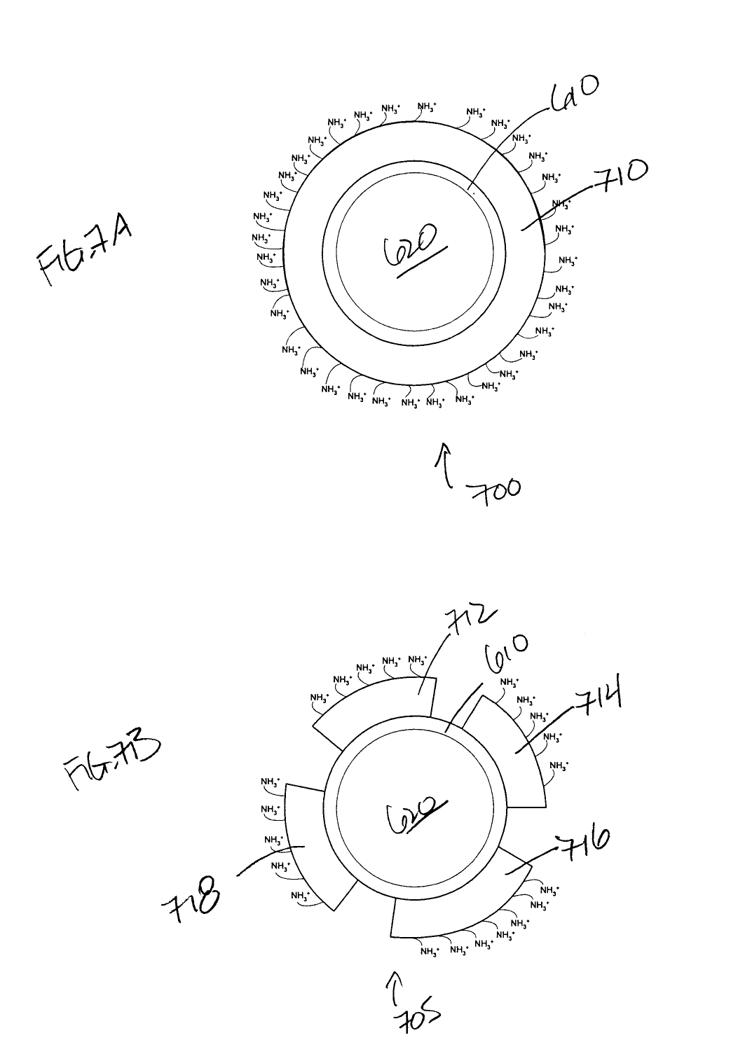

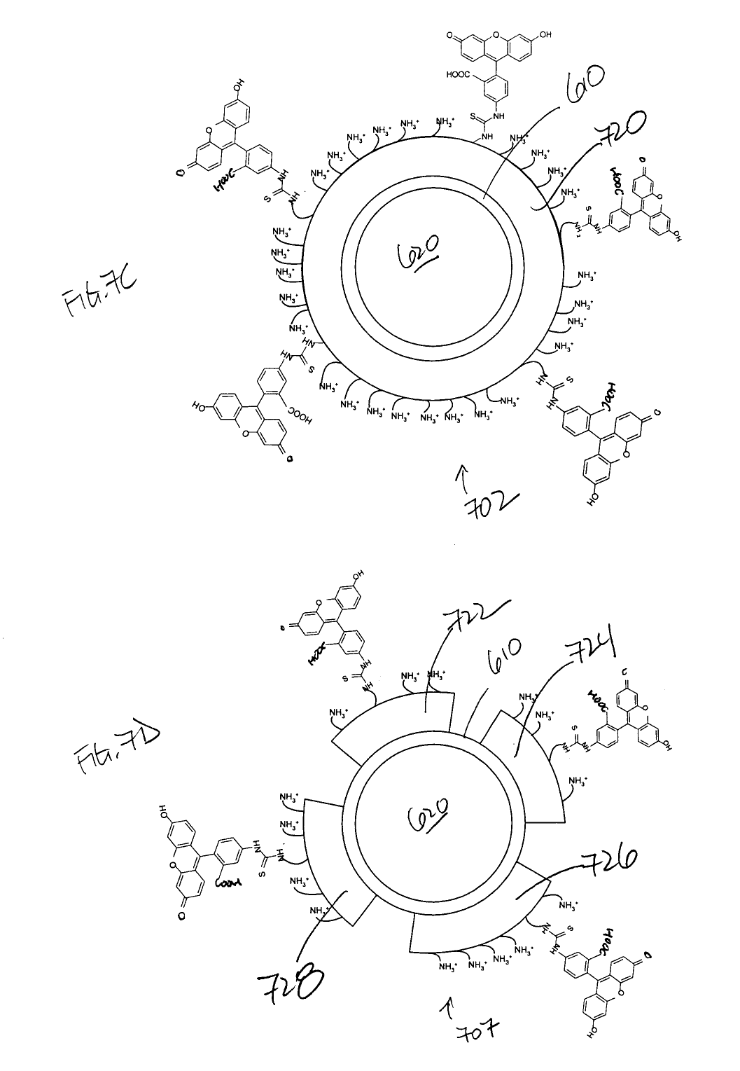

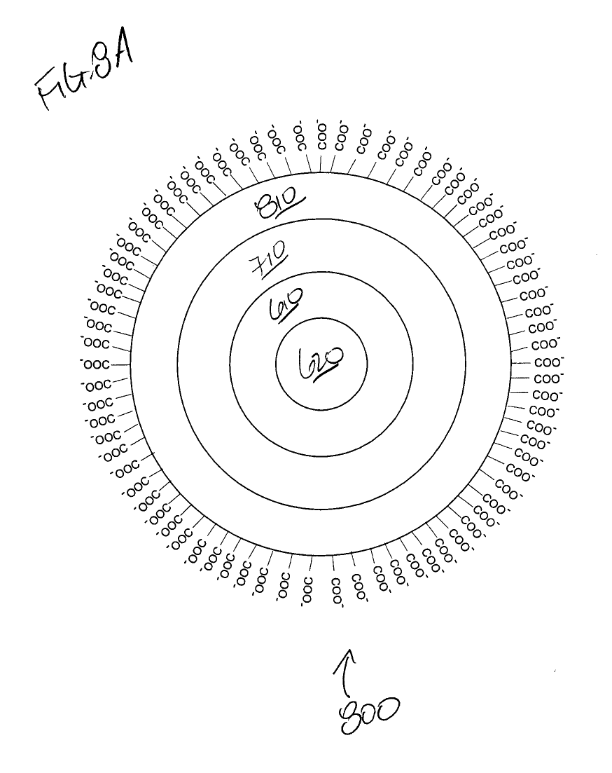

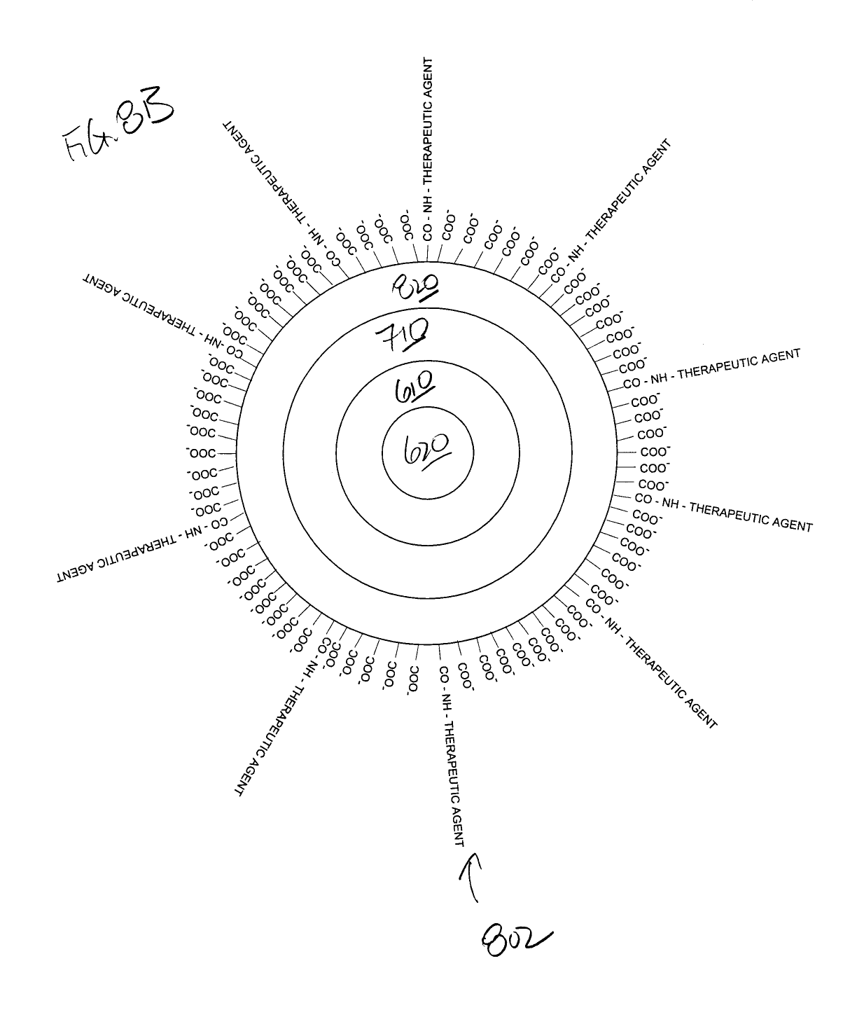

Referring now to FIG. 7A, microsphere 700 comprises Applicant's phospholipids 610 which define internal void 620. In the illustrated embodiment of FIG. 7A, polymeric layer 710 comprising PLL completely encapsulates the spherical phospholipid 610. As those skilled in the art will appreciate, PLL comprises a plurality of pendant amino groups. In the illustrated embodiment of FIG. 7A, those pendant amino groups are shown as ammonium salts. For the sake of clarity and illustration only, the negatively-charged counterions associated with each of the ammonium groups are not shown in FIG. 7A.

Referring now to FIG. 7B, microsphere 705 comprises Applicant's phospholipids 610 defining internal void 620. In the illustrated embodiment of FIG. 7B, PLL portions 712, 714, 716, and 718, partially encapsulate the spherical phospholipid 610. In the illustrated embodiment of FIG. 7B, the pendant amino groups are shown as ammonium salts. For the sake of clarity and illustration only, the negatively-charged counterions associated with each of the ammonium groups are not shown in FIG. 7B.

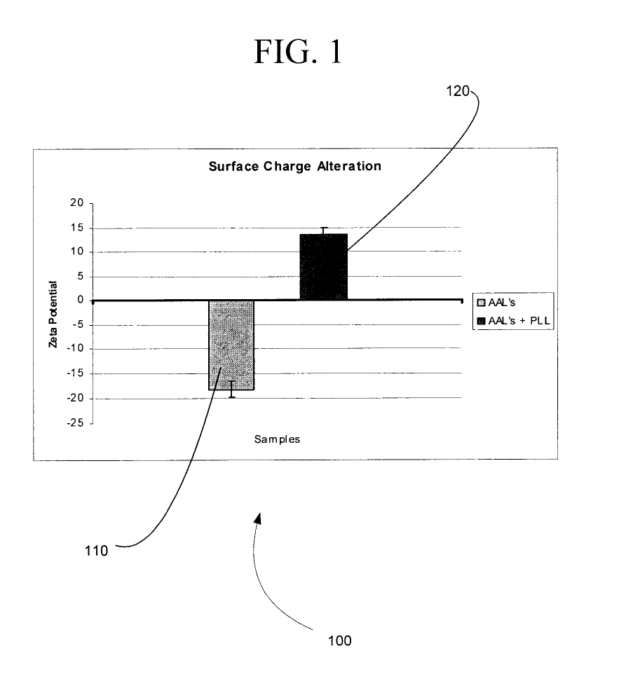

Referring now to FIGS. 1 and 6B, the surface charge of microsphere 600 was analyzed using the Malvern zeta potential, and as shown in curve 110 was determined to be negative at near neutral pH. Referring now to FIGS. 1, 7A, and 7B, a plurality of microspheres 700/705 were then formed by disposing PLL I over part or all of the surface 615 (FIG. 6) of a plurality of microspheres 600 (FIG. 6). As shown in curve 120, microspheres 700/705 comprise a positive surface charge.

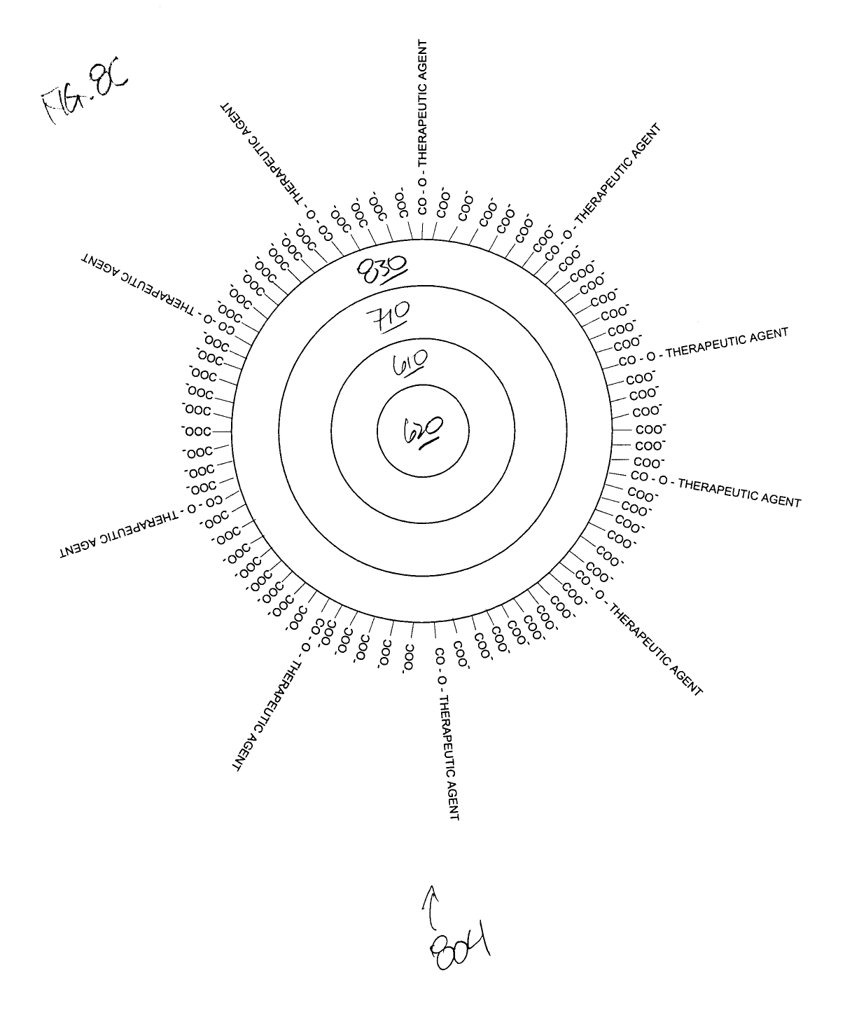

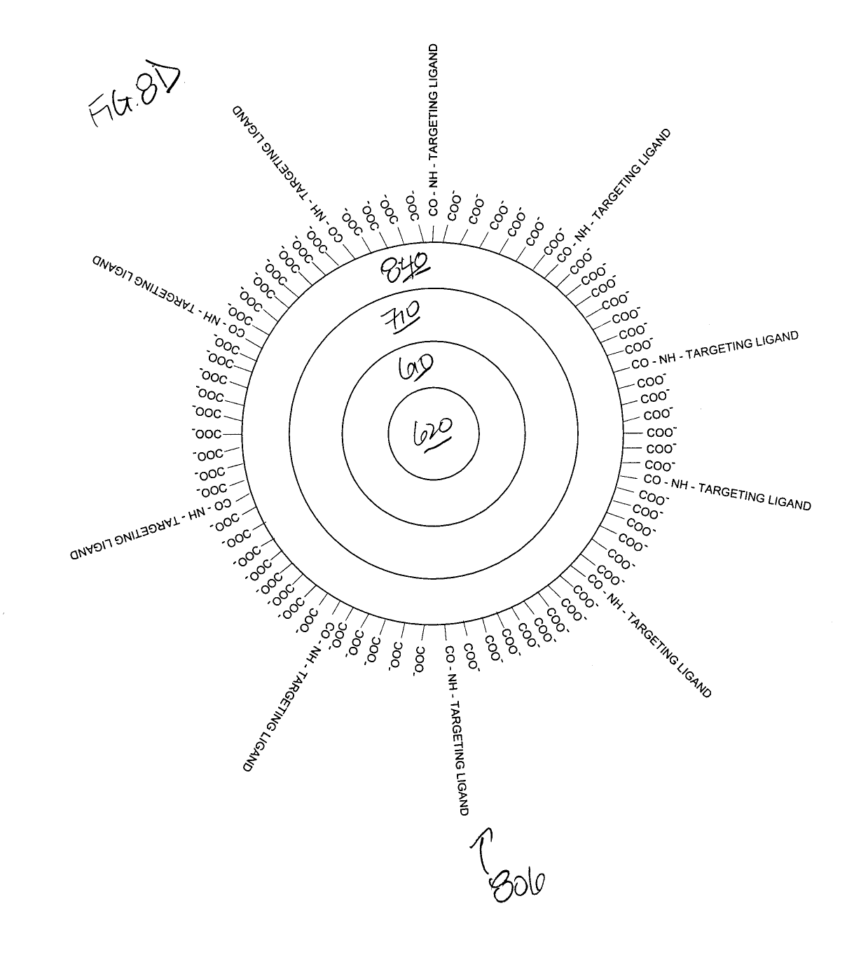

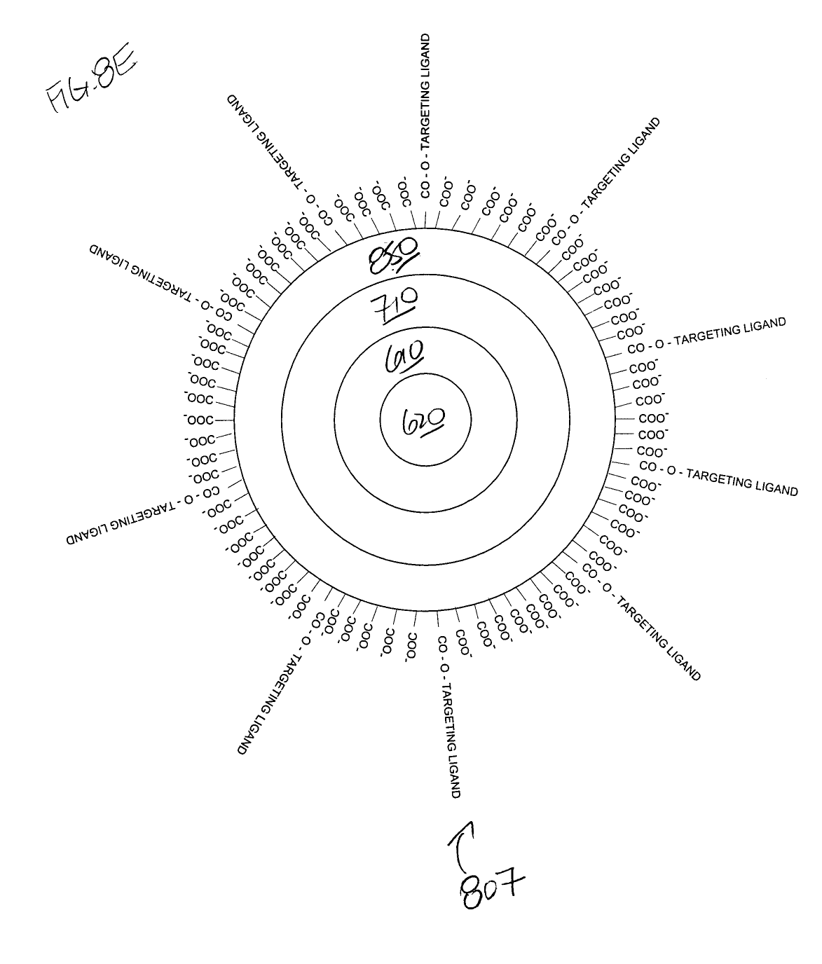

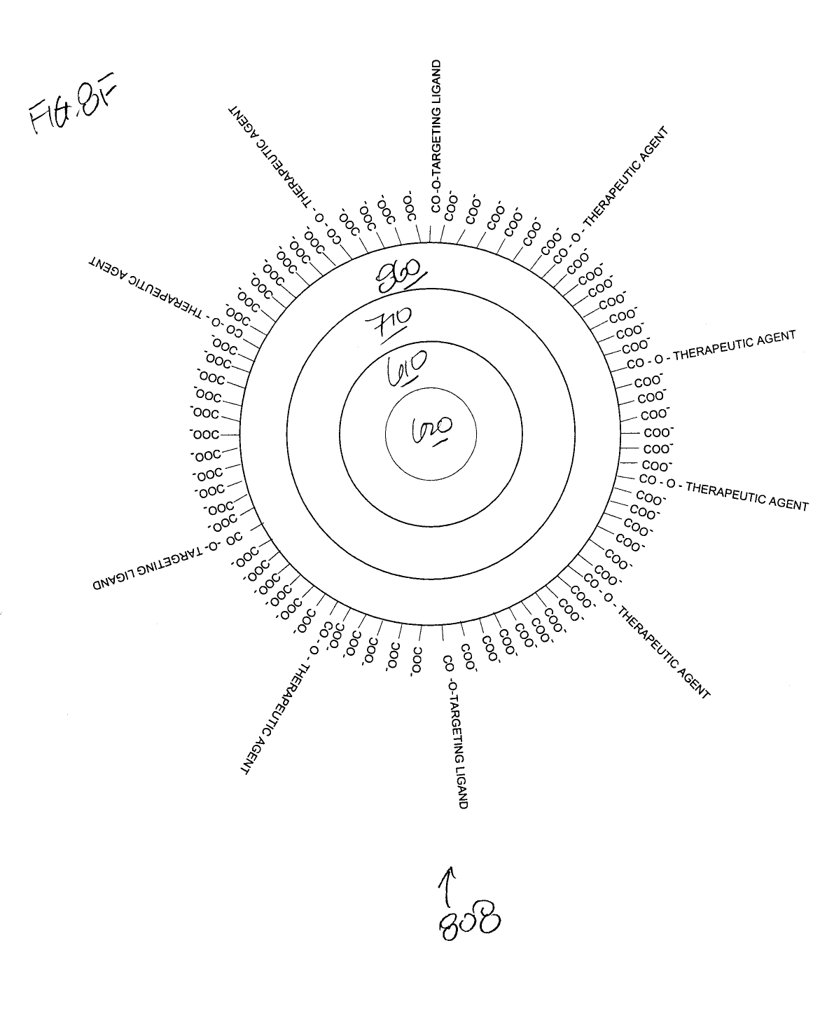

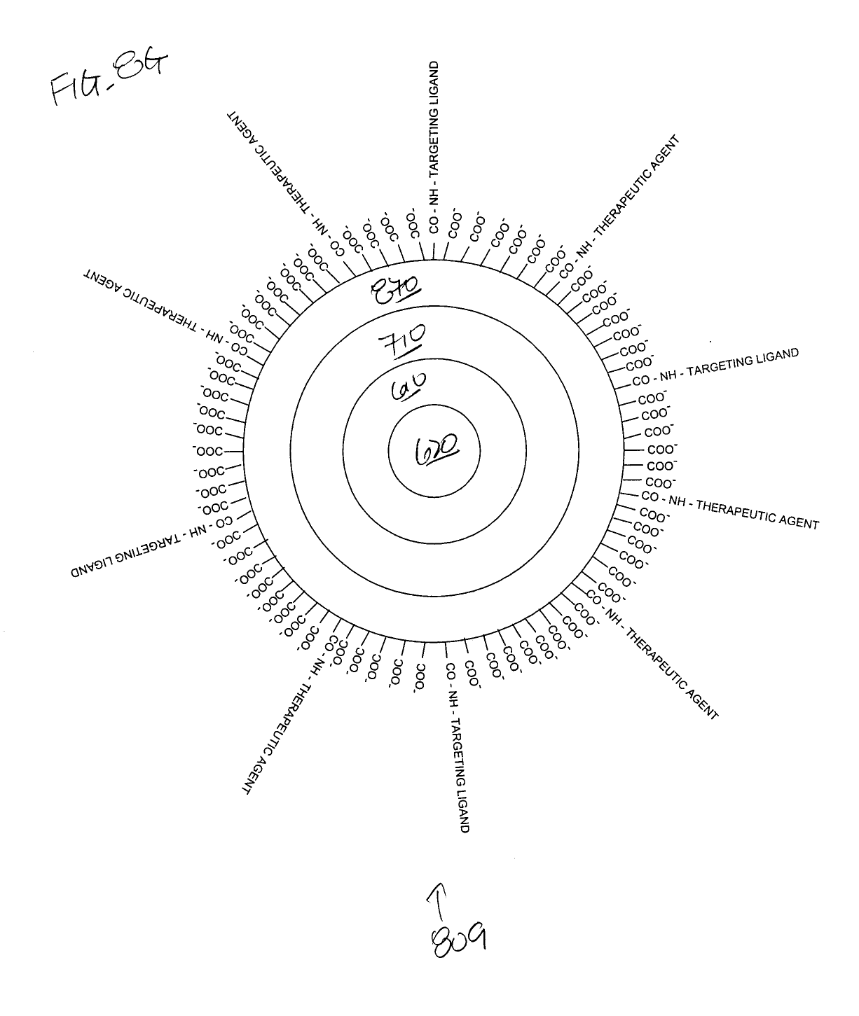

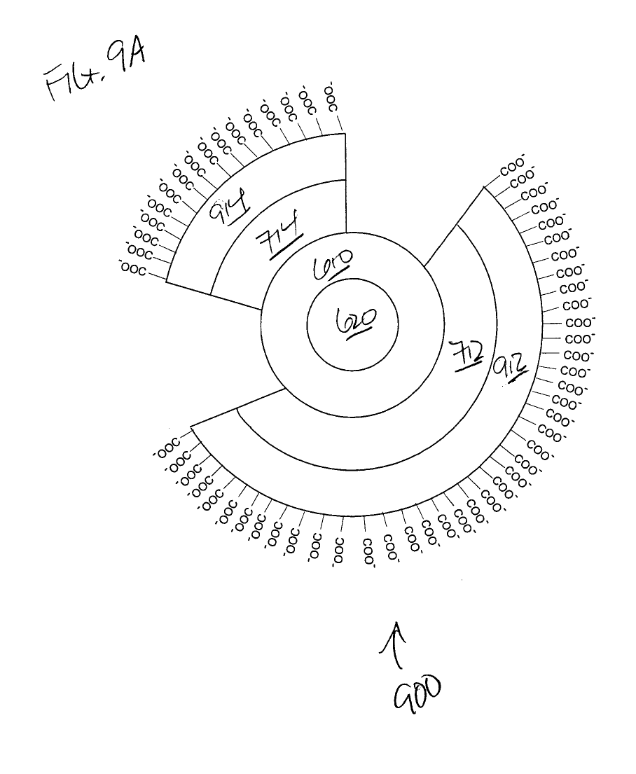

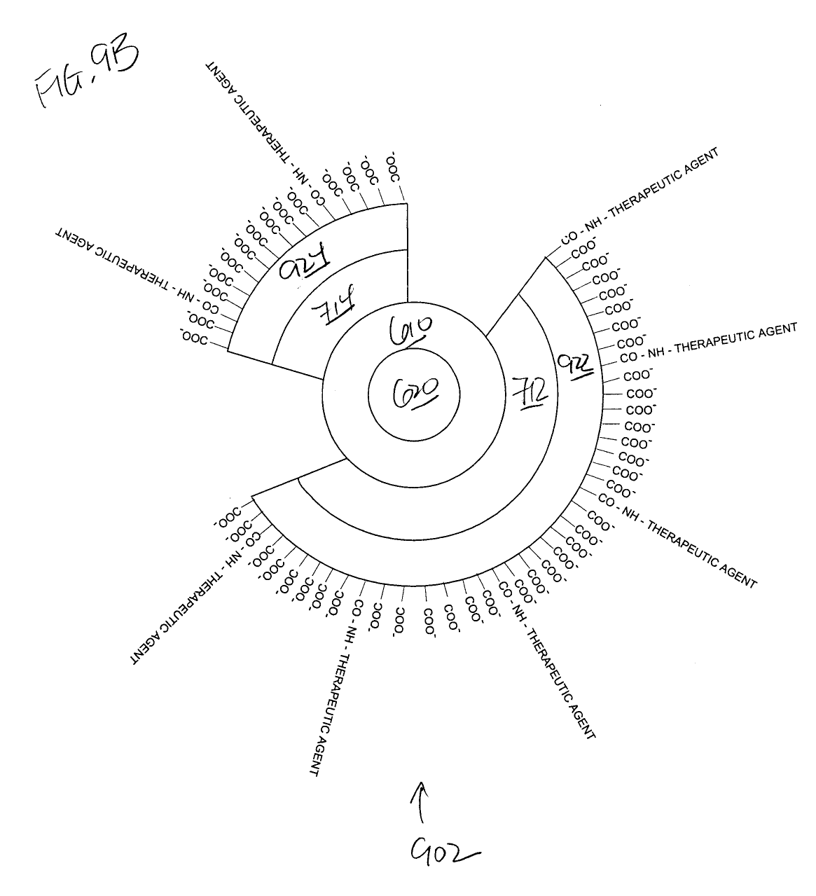

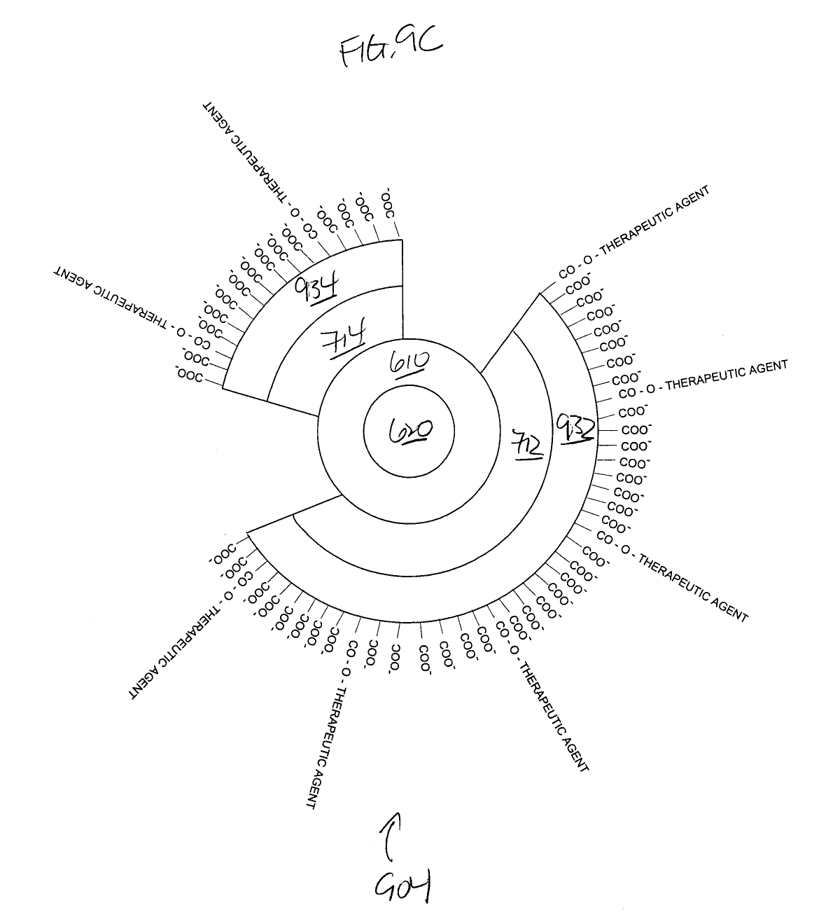

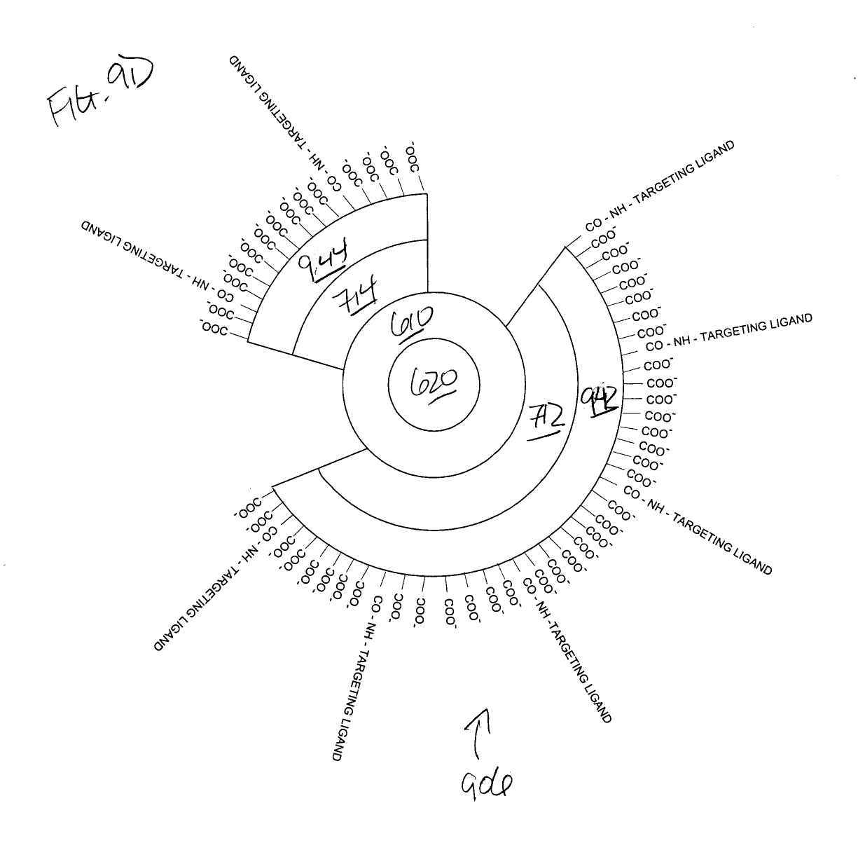

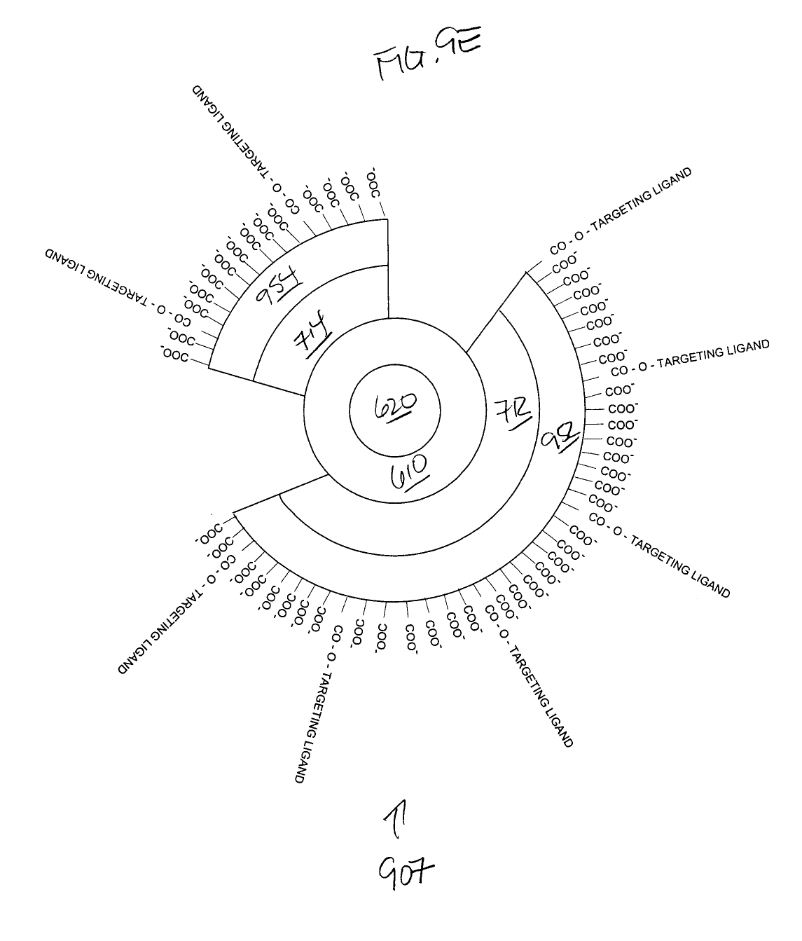

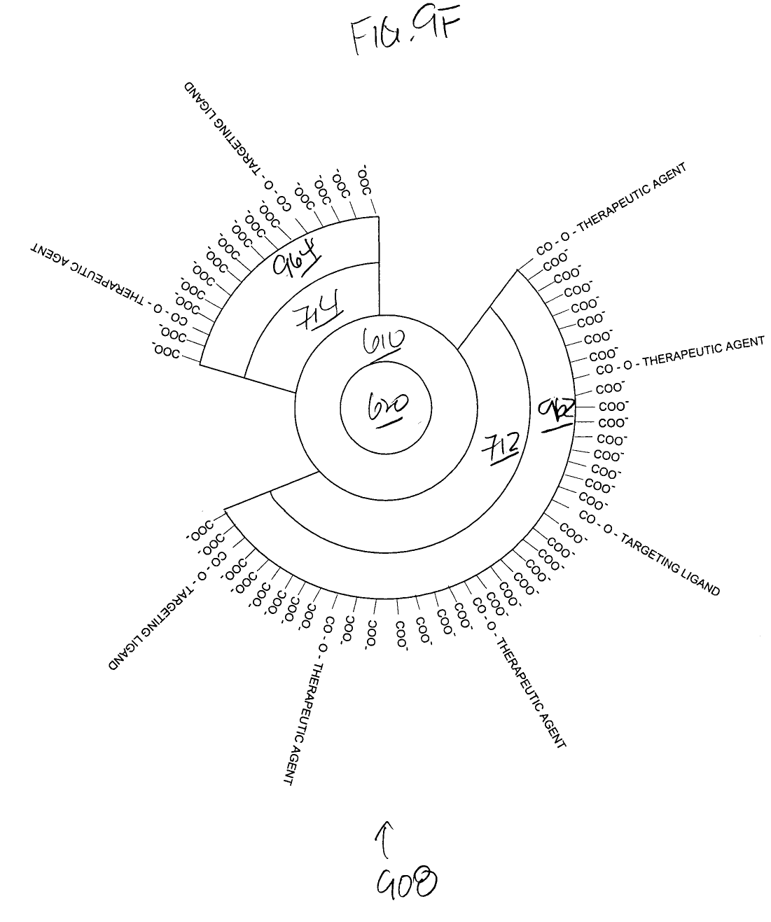

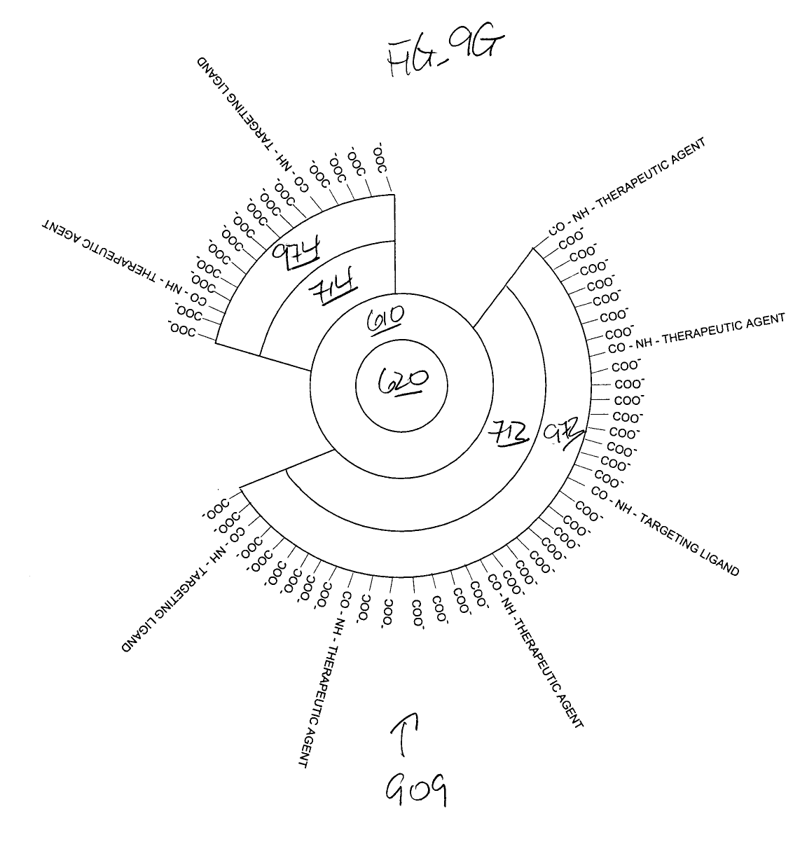

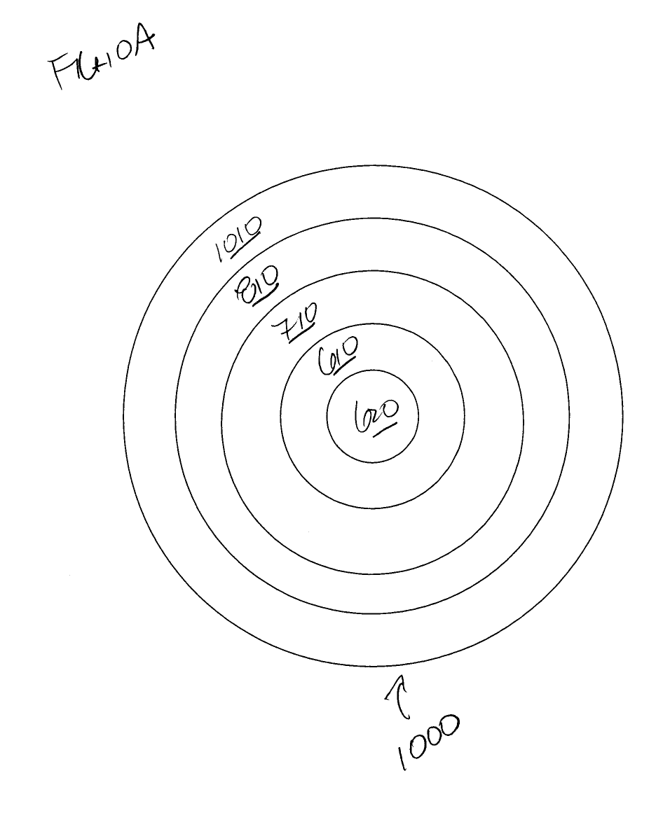

Referring now to FIGS. 8A, and 9A, a second polymeric layer comprising PGA was disposed over all or part of first PLL layer 710 and/or the PLL elements 712 and 714 to give microspheres 800 and/or 900, respectively, wherein layers 810 (FIG. 8A), 912 (FIG. 9A), and 914 (FIG. 9A) comprise PGA II. As those skilled in the art will appreciate, PGA comprises a plurality of pendant carboxylic acid groups. In the illustrated embodiment of FIGS. 8A and 9A, those pendant carboxylic acid groups are shown as carboxylate anions. For the sake of clarity and illustration only, the positively-charged counterions associated with each of the ammonium groups are not shown in FIGS. 8A through 8E, and 9A through 9E.

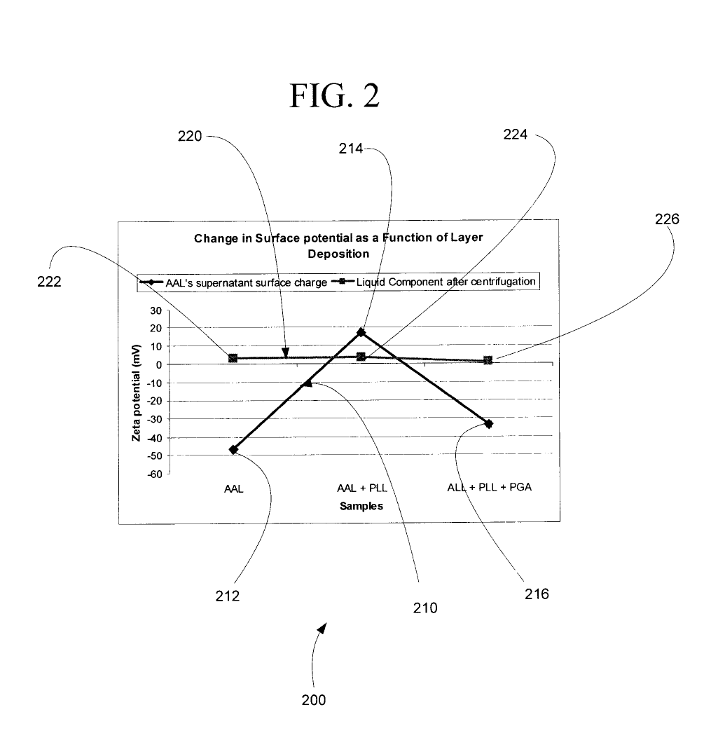

Applicant measured the surface charge of a plurality of microspheres 800/900 using a Malvern zeta potential. Referring now to FIG. 2, curve 210 at point 212 shows the measured potential for a plurality of unmodified microspheres 600 (FIG. 6), at point 214 a plurality of once-modified microspheres 700 (FIG. 7A)/705 (FIG. 7B) comprising a partial and/or a complete encapsulation of a plurality of microspheres 600 with a first polymeric layer of PLL, and at point 216 twice-modified microspheres 800 (FIG. 8A)/900 (FIG. 9A) comprising a partial and/or a complete encapsulation of a plurality of microspheres 600 with a first polymeric layer of PLL in combination with a partial and/or a complete encapsulation of that first polymeric layer by a second polymeric layer PGA.

Curve 210 shows an alternating surface charge regime, wherein the unmodified microspheres 600 comprise a negative surface charge, and wherein the once-modified microspheres 700/705 comprise a positive surface charge, and wherein the twice-modified microspheres 800/900 comprises a negative surface charge. Curve 220 shows a control comprising the measured potential for the liquid component left after sample centrifugation for the unmodified microspheres 600 at point 222, the once-modified microspheres 700/705 at point 224, and the twice-modified microspheres 800/900 at point 226.

In order to show that the negative surface charge measured on the twice-modified microspheres 800/900 results from deposition of a second polymeric layer comprising PGA rather than removal of the first polymeric layer PLL, Applicant attached a fluorophone moiety to a portion of the first polymeric layer PLL. More specifically, Applicant reacted polymer I with fluorescein isothiocynate III to give substituted PLL IV comprising a plurality of pendant fluorescein groups. Those pendant fluorescein groups fluoresce by absorbing UV energy and emitting visible light.

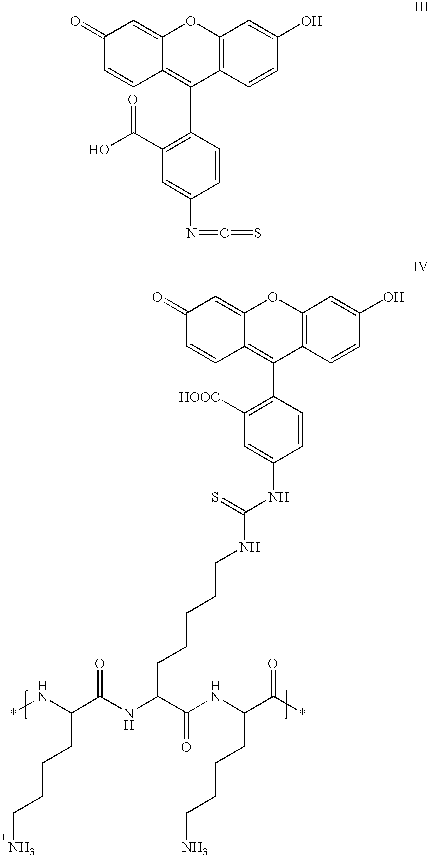

Applicant then disposed fluorescein derivatized PLL IV onto surface 615 of a plurality of microspheres 600 (FIG. 6) to partially and/or completely encapsulate microspheres 600 to give a plurality of microspheres 702 (FIG. 7C) and/or microspheres 707 (FIG. 7D). As those skilled in the art will appreciate, a plurality of microspheres 702/707 will fluoresce under UV irradiation to emit visible light.

Applicant then partially encapsulated microspheres 702/707 with a PGA polymeric layer. As those skilled in the art will further appreciate, those partially encapsulated microspheres 702/707 will also fluoresce under UV irradiation to emit visible light.

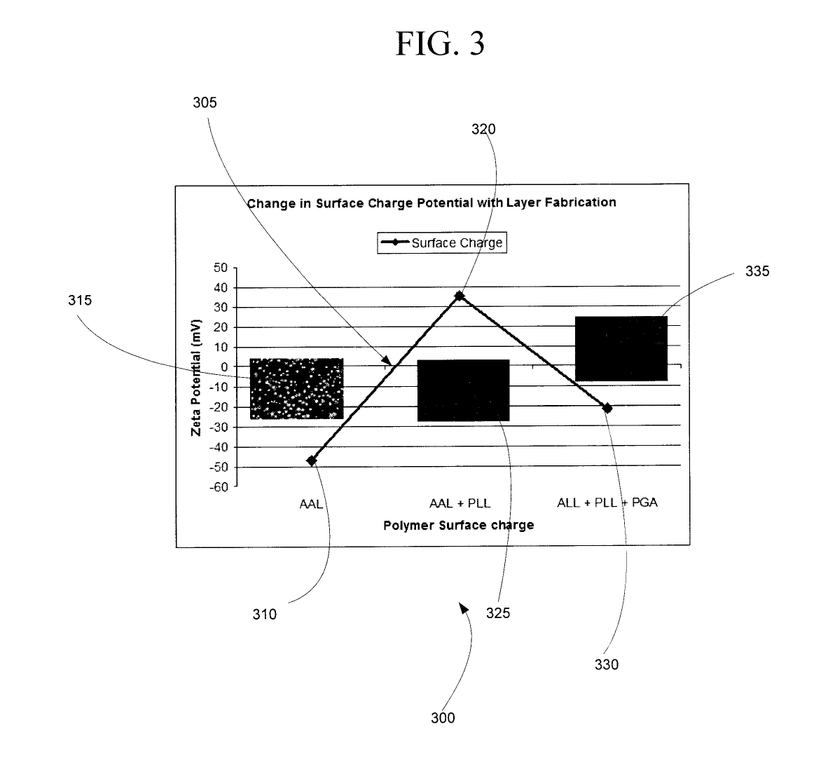

Referring to FIG. 3, micrographs 315, 325, and 335, shows an analysis of unmodified microspheres 600, once-modified microspheres 702/707, and PGA treated microspheres 702/700, respectively, using a fluorescence mode under 60× magnification. The unmodified microspheres 600 did not fluorescence. Fluorescein-derivatized PLL encapsulated microspheres 702/707, and the PGA encapsulated microspheres 702/707, did fluoresce.

Referring now to FIG. 3, curve 310 shows at point 310 the measured potential for unmodified microspheres 600 (FIG. 6B), at point 320 the measured potential for microspheres 702 (FIG. 7C)/707 (FIG. 7D), and at point 320 the measured potential for microspheres 702/707 which are encapsulated with PGA. Microspheres 600 again show a negative surface charge. Microspheres 702/707 show a positive surface charge. Microspheres 702/707 which are encapsulated with PGA show a negative surface charge.

The surface charge data shown in FIG. 310 in combination with the fluorescence data shown in micrographs 315, 325, and 335, demonstrate that Applicant successfully disposed a first polymeric layer over part or all of a plurality of microspheres 600, and can then successfully disposed a second polymer layer over part or all of the first polymeric layer, such that the unmodified microspheres 600 (FIG. 6) comprise a negative surface charge, and such that the once-modified microspheres 702 (FIG. 7A)/707 (FIG. 7B) comprise a positive surface charge, and such that the twice-modified microspheres 800 (FIG. 8A)/900 (FIG. 9A) comprise a negative surface charge.

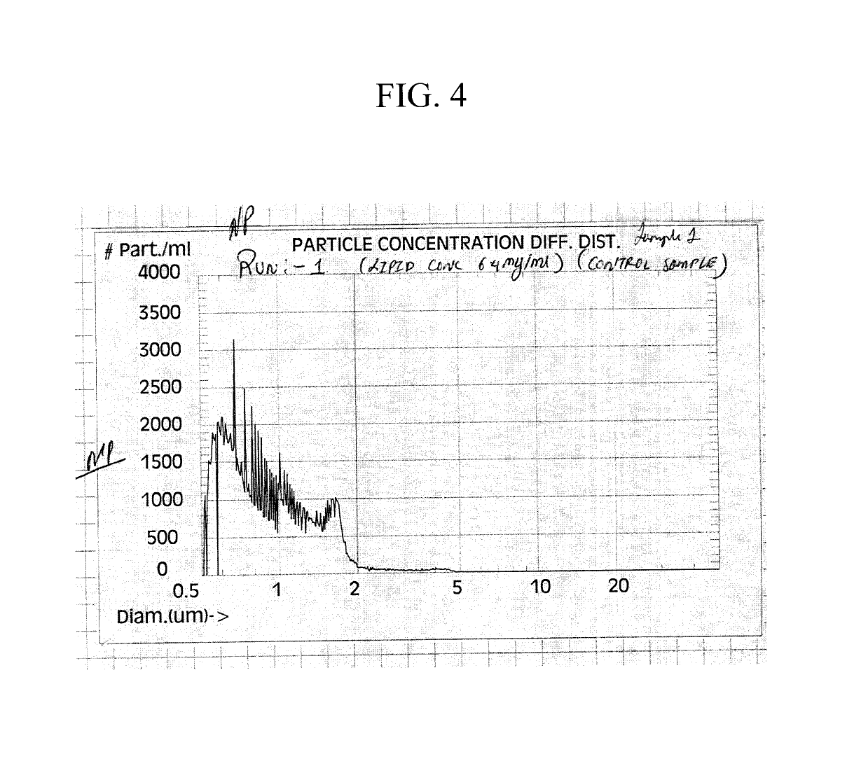

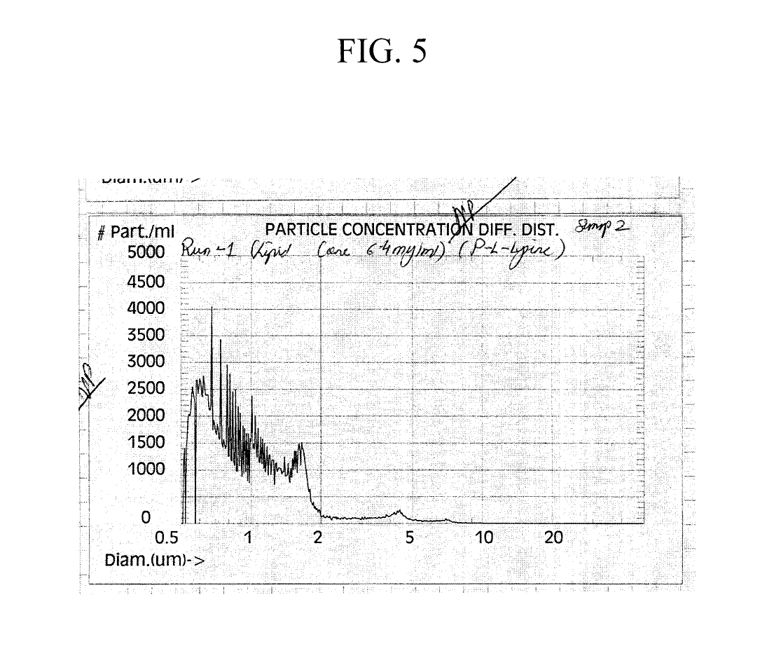

Applicant measured the effects of microsphere size with charge modification. Applicant tested a plurality of unmodified microspheres 600 and a plurality of once-modified microspheres 702/707, with a particle size analyzer. FIG. 4 shows the results for the unmodified microspheres 600. FIG. 5 shows the results for the PLL-modified microspheres 702/707. The data of FIGS. 4 and 5 show no substantial difference in size distribution between the unmodified microspheres 600 and the once-modified microspheres 702/707.

Applicant has found that microspheres 800 (FIG. 8A) and/or 900 (FIG. 9A) can be used to deliver an effective dosage of one or more Therapeutic Agents to a patient in need of those one or more Therapeutic Agent by disposing a plurality of Therapeutic Agent derivatized microspheres 800/900 into that patient by any of the following routes: intraabdominal, intraarterial, intraarticular, intracapsular, intracervical, intracranial, intraductal, intradural, intralesional, intralumbar, intramural, intraocular, intraoperative, intraparietal, intraperitoneal, intrapleural, intrapulmonary, intraspinal, intrathoracic, intratracheal, intratympanic, intrauterine, and intraventricular.

In certain embodiments, Applicant's Therapeutic Agent is selected from the group consisting of one or more camptothecins, one or more taxoids, one or more taxines, one or more taxanes, mimetics of taxol, eleutherobins, sarcodictyins, discodermolides and epothiolones, and combinations thereof. In certain embodiments, Applicant's Therapeutic Agent comprises paclitaxel.

As a general matter, Applicant's Therapeutic Agent comprises any compound natural or synthetic which has a biological activity. This includes peptides, non-peptides and nucleotides.

In certain embodiments, Applicant's Therapeutic Agent(s) comprises any natural or synthetic molecule which is effective against one or more forms of cancer. This definition includes molecules which by their mechanism of action are cytotoxic (anti-cancer agents), those which stimulate the immune system (immune stimulators) and modulators of angiogenesis. The outcome in either case is the slowing of the growth of cancer cells.

In certain embodiments, Applicant's Therapeutic Agent(s) are drawn from the following list: Taxotere, Amonafide, Illudin S, 6-hydroxymethylacylfulvene Bryostatin 1, 26-succinylbryostatin 1, Palmitoyl Rhizoxin, DUP 941, Mitomycin B, Mitomycin C, Penclomedine. Interferon angiogenesis inhibitor compounds, Cisplatin hydrophobic complexes such as 2-hydrazino-4,5-dihydro-1H-imidazole with platinum chloride and 5-hydrazino-3,4-dihydro-2H-pyrrole with platinum chloride, vitamin A, vitamin E and its derivatives, particularly tocopherol succinate.

In certain embodiments, Applicant's Therapeutic Agent(s) comprises 1,3-bis(2-chloroethyl)-1-nitrosurea (“carmustine” or “BCNU”), 5-fluorouracil, doxorubicin (“adriamycin”), epirubicin, aclarubicin, Bisantrene (bis(2-imidazolen-2-ylhydrazone)-9,10-anthracenedicarboxaldehyde, mitoxantrone, methotrexate, edatrexate, muramyl tripeptide, muramyl dipeptide, lipopolysaccharides, 9-b-d-arabinofuranosyladenine (“vidarabine”) and its 2-fluoro derivative, resveratrol, retinoic acid and retinol, Carotenoids, and tamoxifen.

In certain embodiments, Applicant's Therapeutic Agent(s) comprises Palmitoyl Rhizoxin, DUP 941, Mitomycin B, Mitomycin C, Penclomedine, Interferon .alpha.2b, Decarbazine, Lonidamine, Piroxantrone, Anthrapyrazoles, Etoposide, Camptothecin, 9-aminocamptothecin, 9-nitrocamptothecin, camptothecin-11 (“Irinotecan”), Topotecan, Bleomycin, the Vinca alkaloids and their analogs [Vincristine, Vinorelbine, Vindesine, Vintripol, Vinxaltine, Ancitabine], 6-aminochrysene, and navelbine.

In certain embodiments, the one or more Therapeutic Agents described hereinabove can by administered to a patient in need thereof by attaching those one or more Therapeutic Agents to Applicants' microspheres 800 (FIG. 8A) and/or microspheres 900 (FIG. 9A). In certain embodiments, Applicant reacts PGA II with amino-derivatized Therapeutic Agent V to form Therapeutic Agent-derivatized PGA VI, wherein (n) is between 50 and 300, and then disposes that Therapeutic Agent-derivatized PGA VI onto microspheres 702 (FIG. 7A) and/or microspheres 707 (FIG. 7B) to form Therapeutic Agent derivatized microspheres 802 (FIG. 8B) and/or Therapeutic Agent derivatized microspheres 902 (FIG. 9B), wherein layers 820 (FIG. 8B), 922 (FIG. 9B), and 924 (FIG. 9B), comprise Therapeutic Agent-derivatized PGA VI.

In certain embodiments, Applicant reacts PGA II with hydroxyl-derivatized Therapeutic Agent VII to form Therapeutic Agent-derivatized PGA VIII, wherein (n) is between 50 and 300, and then disposes that Therapeutic Agent-derivatized PGA VIII onto microspheres 702 (FIG. 7A) and/or microspheres 707 (FIG. 7B) to form Therapeutic Agent derivatized microspheres 804 (FIG. 8C) and/or Therapeutic Agent derivatized microspheres 904 (FIG. 9C), wherein layers 830 (FIG. 8C), 932 (FIG. 9C), and 934 (FIG. 9C), comprises Therapeutic Agent-derivatized PGA VIII.

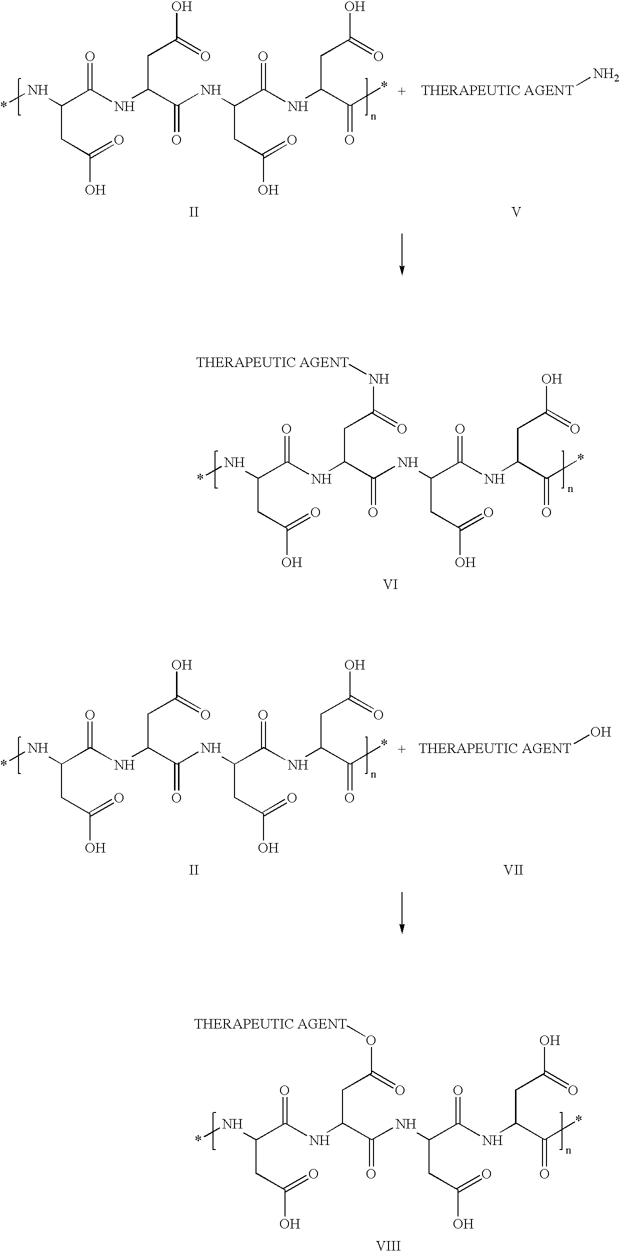

Applicant has found that attached a targeting ligand to a plurality of microspheres 800 (FIG. 8A) and/or to a plurality of microspheres 900 (FIG. 9A) is useful to direct those microspheres to a selected target site to facilitate a treatment protocol using ultrasound energy. Such as target site may comprise, for example and without limitation, a thrombus, a carcinoma, and the like.

In certain embodiments, Applicant reacts PGA II with amino-derivatized Targeting Ligand IX to form Targeting Ligand-derivatized PGA X, wherein (n) is between 50 and 300, and then disposes that Targeting Ligand-derivatized PGA X onto microspheres 702 (FIG. 7A) and/or microspheres 707 (FIG. 7B) to form Targeting Ligand derivatized microspheres 806 (FIG. 8D) and/or Targeting Ligand derivatized microspheres 906 (FIG. 9D), wherein layers 840 (FIG. 8D), 942 (FIG. 9D), and 944 (FIG. 9D), comprise Targeting Ligand-derivatized PGA X.

In certain embodiments, Applicant reacts PGA II with hydroxyl-derivatized Targeting Ligand XI to form Targeting Ligand-derivatized PGA XII, wherein (n) is between 50 and 300, and then disposes that Targeting Ligand-derivatized PGA XII onto microspheres 702 (FIG. 7A) and/or microspheres 707 (FIG. 7B) to form Targeting Ligand derivatized microspheres 807 (FIG. 8E) and/or Targeting Ligand derivatized microspheres 907 (FIG. 9E), wherein layers 850 (FIG. 8E), 952 (FIG. 9E), and 954 (FIG. 9E), comprise Targeting Ligand-derivatized PGA XII.

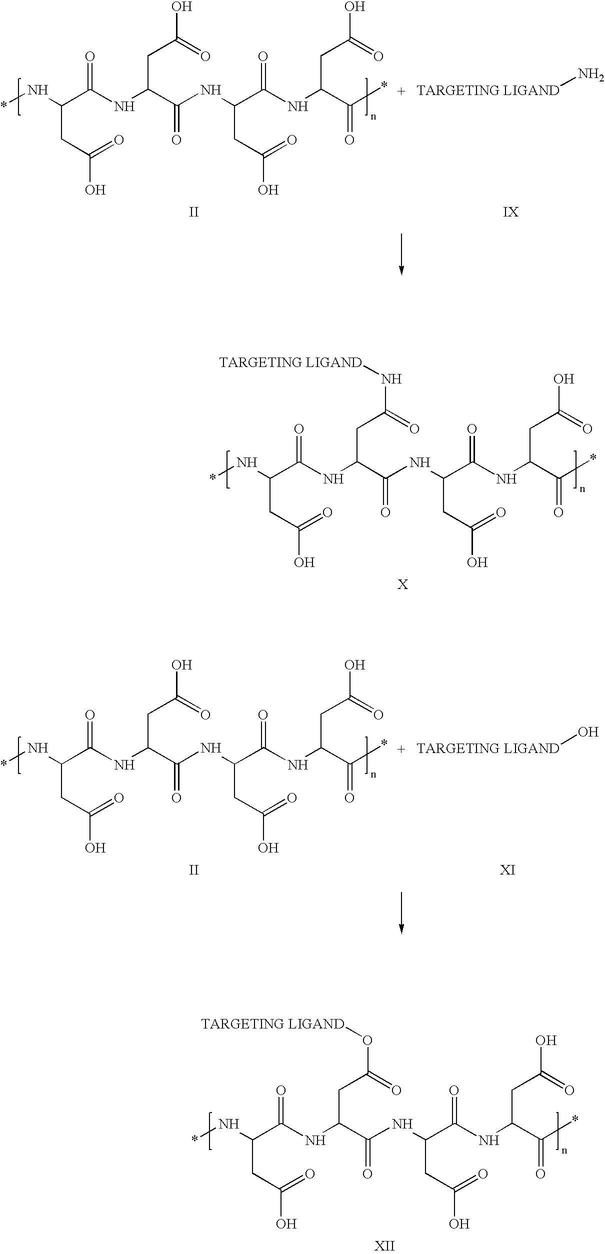

In certain embodiments, Applicant further derivatives Therapeutic Agent-derivatized PGA VIII or Targeting Ligand-derivatized PGA XII to give Therapeutic Agent & Targeting Ligand derivatized PGA XIII comprising a PGA backbone in combination with one or more pendant Targeting Ligands and one or more pendant Therapeutic Agents.

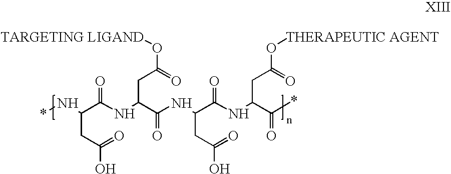

Applicant then disposes Therapeutic Agent & Targeting Ligand derivatized PGA XIII onto microspheres 702 (FIG. 7A) and/or microspheres 707 (FIG. 7B) to form Therapeutic Agent & Targeting Ligand derivatized microspheres 808 (FIG. 8F) and/or Therapeutic Agent & Targeting Ligand derivatized microspheres 908 (FIG. 9F), wherein layers 860 (FIG. 8F), 962 (FIG. 9F), and 964 (FIG. 9F), comprise Therapeutic Agent & Targeting Ligand derivatized PGA XIII.

In certain embodiments, Applicant further derivatizes Therapeutic Agent-derivatized PGA VI or Targeting Ligand-derivatized PGA X to give Therapeutic Agent & Targeting Ligand derivatized PGA XIV comprising a PGA backbone in combination with one or more pendant Targeting Ligands and one or more pendant Therapeutic Agents.

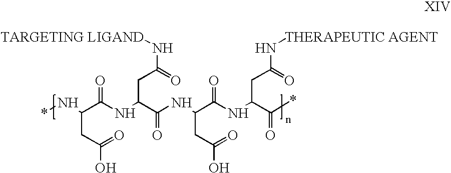

Applicant then disposes Therapeutic Agent & Targeting Ligand derivatized PGA XIV onto microspheres 702 (FIG. 7A) and/or microspheres 707 (FIG. 7B) to form Therapeutic Agent & Targeting Ligand derivatized microspheres 809 (FIG. 8G) and/or Therapeutic Agent & Targeting Ligand derivatized microspheres 909 (FIG. 9G), wherein layers 870 (FIG. 8G), 972 (FIG. 9G), and 974 (FIG. 9G), comprise Therapeutic Agent & Targeting Ligand derivatized PGA XIV.

In certain embodiments, Applicant disposes a colloidal gel onto microsphere 700 (FIG. 7A), microsphere 705 (FIG. 7B), microsphere 800 (FIG. 8A) and/or microsphere 900 (FIG. 9A).

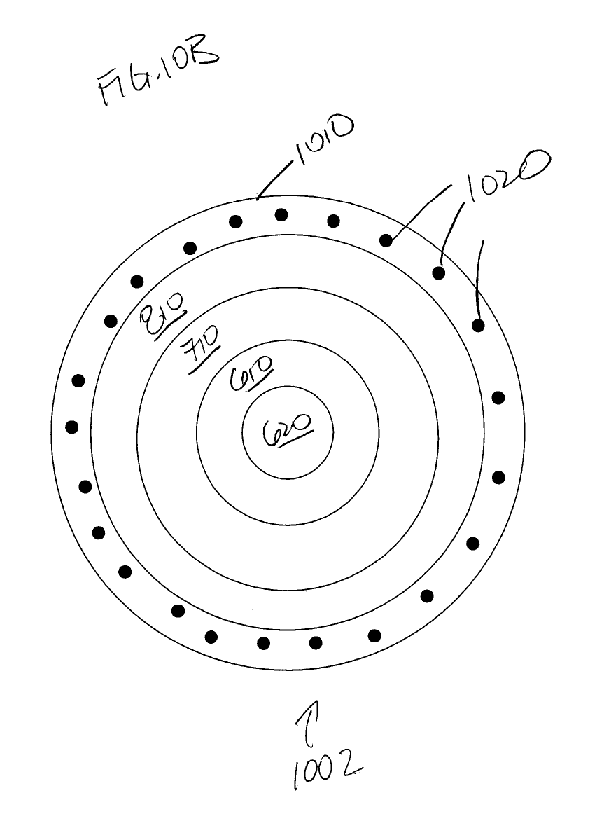

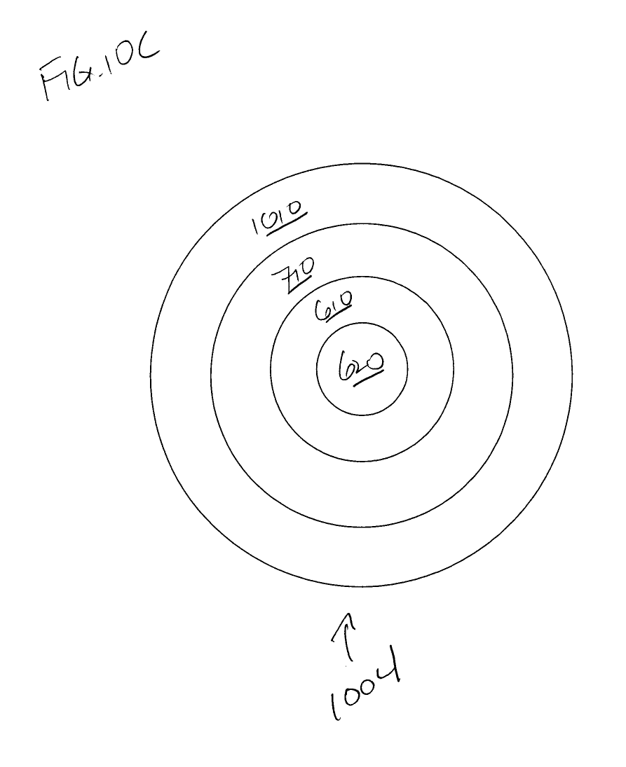

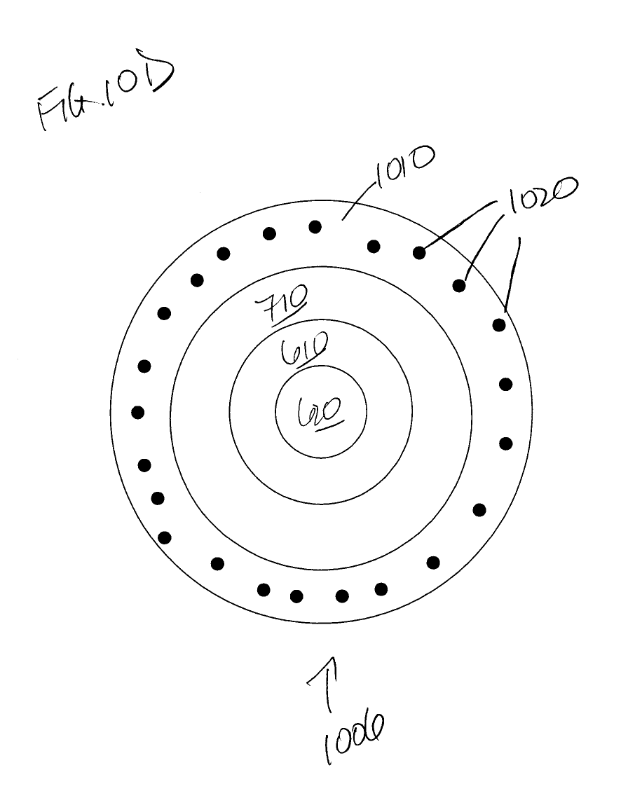

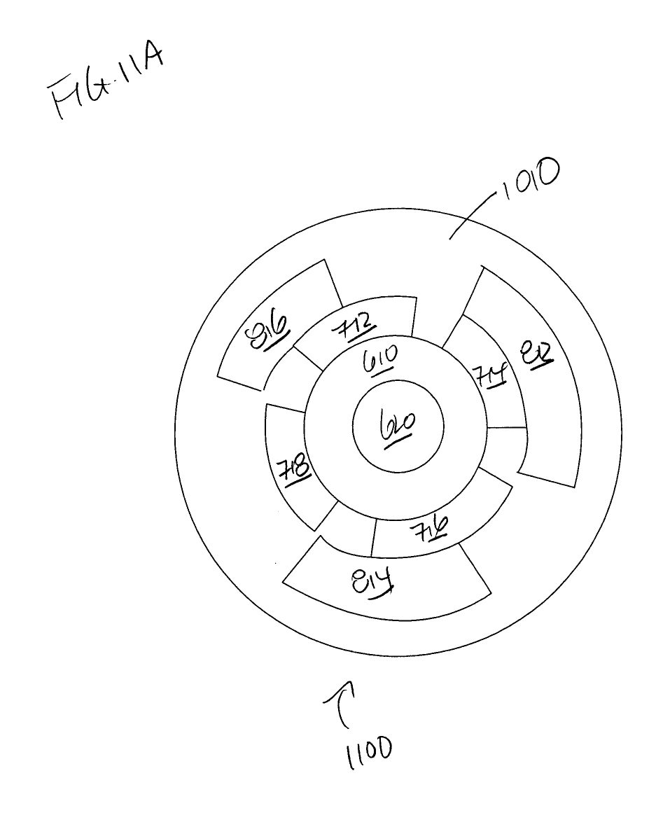

Referring now to FIG. 10A, microsphere 1 000 comprises microsphere 600 (FIGS. 6A, 6B) which comprises one or more phospholipid compounds 610, wherein those one or more phospholipids define internal void 620, a first polymeric layer 710 encapsulating microsphere 600, a second polymeric layer 810 encapsulating the first polymeric layer 710, and colloidal gel 1010 encapsulating the second polymeric layer 810.

Referring now to FIG. 10C, microsphere 1004 comprises microsphere 600 (FIGS. 6A, 6B) which comprises one or more phospholipid compounds 610, wherein those one or more phospholipids define internal void 620, a first polymeric layer 710 encapsulating microsphere 600, and colloidal gel 1010 encapsulating the first polymeric layer 710.

In certain embodiments, colloidal gel 1010 comprises agar. In certain embodiments, colloidal gel 1010 comprises gelatin dispersed in water. By “gelatin” Applicant means a protein product produced by partial hydrolysis of collagen.

In certain embodiments, Applicant's colloidal gel comprises a hydrogel. By “hydrogel,” Applicant means a network of polymer chains that are at least partially water-soluble, disposed in a aqueous medium.

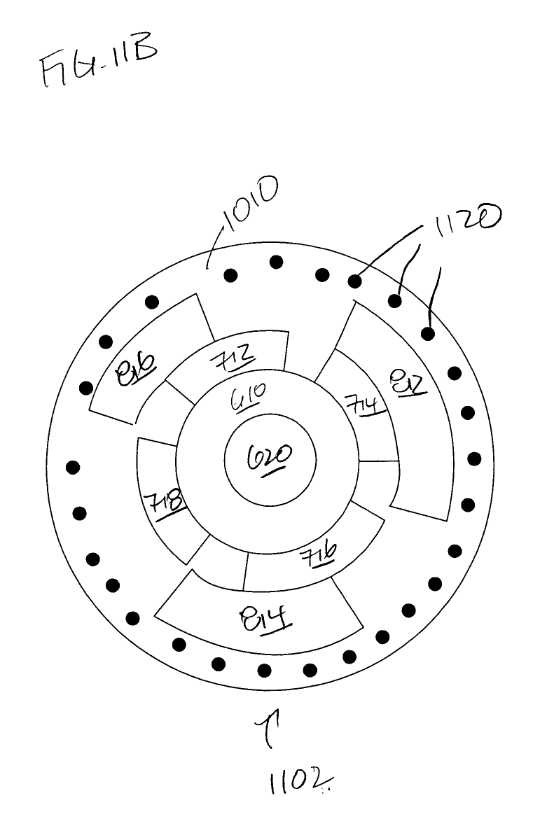

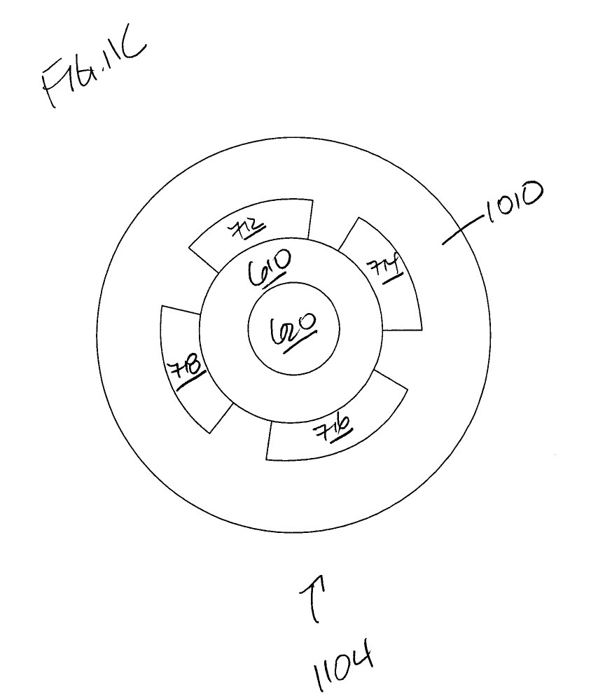

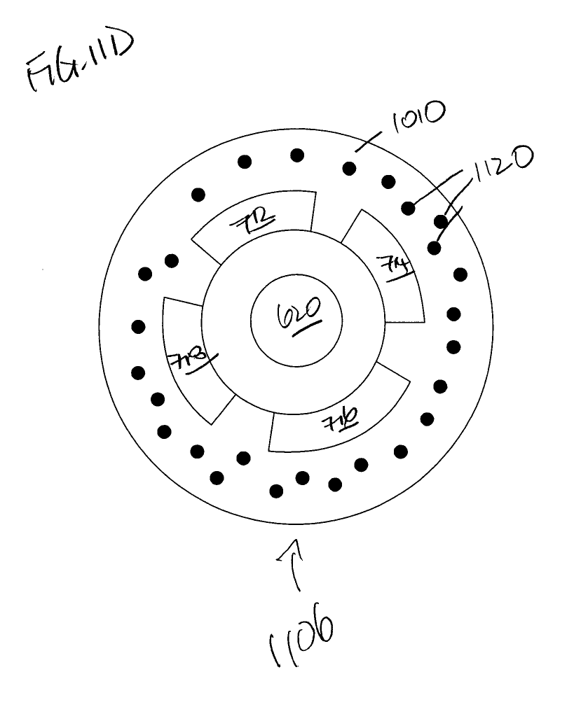

Referring now to FIG. 11A, microsphere 1100 comprises microsphere 600 (FIGS. 6A, 6B) which comprises one or more phospholipid compounds 610, wherein those one or more phospholipids define internal void 620, first polymeric layer portions 712, 714, 716, and 718, each partially encapsulating microsphere 600, second polymeric layer portions 812, 814, and 816, each partially encapsulating the first polymeric layer portions 712, 714, 716, and 718, and colloidal gel 1010 encapsulating the second polymeric layer portions 812, 814, and 816, wherein colloidal gel layer 1010 further encapsulates any exposed portions of first polymeric layer 710, and wherein colloidal gel layer 1010 further encapsulates any exposed portions of microsphere 600.

Referring now to FIG. 11C, microsphere 1104 comprises microsphere 600 (FIGS. 6A, 6B) which comprises one or more phospholipid compounds 610, wherein those one or more phospholipids define internal void 620, first polymeric layer portions 712, 714, 716, and 718, each partially encapsulating microsphere 600, and colloidal gel 1010 encapsulating first polymeric layer portions 712, 714, 716, and 718, and wherein colloidal gel layer 1010 further encapsulates any exposed portions of microsphere 600.

Applicant has found that microspheres 1000, 1004, 1100, and 1104, comprise enhanced stability, and therefore, will likely show enhanced lifetimes when disposed in an animal's, including a human's, circulatory system. In addition, Applicant has found that microsphere 1000, microsphere 1004, microsphere 1100 and/or microsphere 1104, can function as a sustained release vehicle for one or more Therapeutic Agents, as described hereinabove. Referring to FIGS. 10B and 10D, sustained release microspheres 1002 and 1006 comprises microsphere 1000 and microsphere 1004, respectively, wherein one or more discrete domains 1020 comprising one or more Therapeutic Agents are releaseably disposed within a contiguous colloidal gel layer 1010.

Referring to FIGS. 11B and 11D, sustained release microsphere 1102 and 1106 comprises microsphere 1100 and 1104, respectively, wherein one or more discrete domains 1120 comprising one or more Therapeutic Agents are releaseably disposed within a contiguous colloidal gel layer 1010.

Applicant has found that depending on the concentrations of the one or more Therapeutic Agents comprising the one or more discrete domains 1020/1120, and depending on the thickness of colloidal gel layer 1010, and depending on the compatibility between colloidal gel 1010 and the one or more Therapeutic Agents comprising the one or more discrete domains 1020/1120, respectively, the one or more Therapeutic Agents diffuse through colloidal gel 1010 to the surface thereof, and are released from colloidal gel 1010, over a period time ranging from minutes to hours.

In certain embodiments, the one or more Therapeutic Agents are disposed in Applicant's colloidal gel to form the one or more discrete domains, and that colloidal gel comprising one or more discrete domains comprising one or more Therapeutic Agents is then used form microsphere 1002, and/or microsphere 1006, and/or microsphere 1102, and/or microsphere 1106. In other embodiments, Applicant forms microsphere 1000, and/or microsphere 1004, and/or microsphere 1100, and/or microsphere 1104, and then immerses a plurality of microspheres 1000, and/or a plurality of microspheres 1004, and/or a plurality of microspheres 1100, and/or a plurality of microspheres 1104, in an aqueous suspension of one or more Therapeutic Agents, wherein those one or more Therapeutic Agents are more compatible with colloidal gel 1010 than with the water such that those one or more Therapeutic Agents move from the aqueous suspension into the colloidal gel to form microspheres 1002, and/or microspheres 1006, and/or microspheres 1102, and/or microspheres 1106.

While the preferred embodiments of the present invention have been illustrated in detail, it should be apparent that modifications and adaptations to those embodiments may occur to one skilled in the art without departing from the scope of the present invention.

I claim:

1. A polymer-encapsulated microsphere, comprising:
a plurality of lipid molecules disposed in a liquid and arranged to define an enclosed space;
a first polymer partially or completely encapsulating said enclosed space.

2. The polymer-encapsulated microsphere of claim 1, wherein:
said plurality of lipid molecules define a contiguous surface;
said first polymer partially or completely encapsulates said contiguous surface.

3. The polymer-encapsulated microsphere of claim 1 further comprising a gas disposed within said enclosed space.

4. The polymer-encapsulated microsphere of claim 3, wherein said gas is selected from the group consisting of a fluorine-containing gas, a perfluorocarbon gas, sulfur hexafluoride, and mixtures thereof.

5. The polymer-encapsulated microsphere of claim 4, wherein said gas is selected from the group consisting of perfluoropropane and perfluorobutane.

6. The polymer-encapsulated microsphere of claim 1, wherein said plurality of lipids comprise a plurality of phosphorus-containing compounds.

7. The polymer-encapsulated microsphere of claim 6, wherein said plurality of phosphorus-containing compounds comprise dipalmitoylphosphatidic acid, dipalmitoylphosphatidylethanolaminepolyethylene glycol, and dipalmitoylphosphatidylcholine.

8. The polymer-encapsulated microsphere of claim 6, wherein said first polymer comprises Poly/Lysine.

9. A polymer-encapsulated microsphere, comprising:
a plurality of lipid molecules disposed in a liquid and arranged to define an enclosed space;
a first polymer partially or completely encapsulating said enclosed space;
a second polymer partially or completely encapsulating said enclosed space.

10. The polymer-encapsulated microsphere of claim 9, wherein said second polymer partially encapsulates said first polymer.

11. The polymer-encapsulated microsphere of claim 10, wherein said second polymer completely encapsulates said first polymer.

12. The polymer-encapsulated microsphere of claim 9 further comprising a gas disposed within said enclosed space.

13. The polymer-encapsulated microsphere of claim 12, wherein said gas is selected from the group consisting of a fluorine-containing gas, a perfluorocarbon gas, sulfur hexafluoride, and mixtures thereof.

14. The polymer-encapsulated microsphere of claim 13, wherein said gas is selected from the group consisting of perfluoropropane and perfluorobutane.

15. The polymer-encapsulated microsphere of claim 9, wherein said plurality of lipids comprise a plurality of phosphorus-containing compounds.

16. The polymer-encapsulated microsphere of claim 15, wherein said plurality of phosphorus-containing compounds comprise dipalmitoylphosphatidic acid, dipalmitoylphosphatidylethanolaminepolyethylene glycol, and dipalmitoylphosphatidylcholine.

17. The polymer-encapsulated microsphere of claim 9, wherein said first polymer comprises Poly/Lysine.

18. The polymer-encapsulated microsphere of claim 17, wherein said second polymer comprises Poly Glutamic Acid.

19. The polymer-encapsulated microsphere of claim 17, wherein said second polymer comprises the polymer: 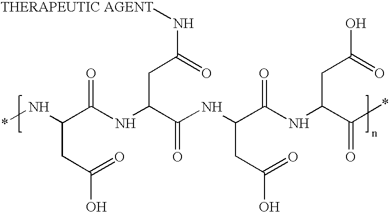
wherein said therapeutic agent is selected from the group consisting of one or more camptothecins, one or more taxoids, one or more taxines, one or more taxanes, mimetics of taxol, eleutherobins, sarcodictyins, discodermolides and epothiolones, and combinations thereof;
wherein n is between about 50 and about 300.

20. The polymer-encapsulated microsphere of claim 17, wherein said second polymer comprises the polymer 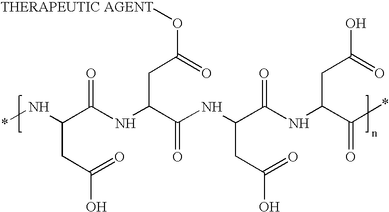
wherein said therapeutic agent is selected from the group consisting of one or more camptothecins, one or more taxoids, one or more taxines, one or more taxanes, mimetics of taxol, eleutherobins, sarcodictyins, discodermolides and epothiolones, and combinations thereof;
wherein n is between about 50 and about 300.
